(19) United States
(12) Patent Application Publication
(10) Pub. No.: US 2018/0075480 A1
(43) Pub. Date: Mar. 15, 2018

(54) METHODS AND SYSTEMS FOR CARRYING OUT VARIOUS TREATMENTS

(71) Applicant: DANIÈLE HENKEL TO GO, Pierrefonds (CA)

(72) Inventors: Danièle HENKEL, Pierrefonds (CA); Linda MAHIEDDINE, Pierrefonds (CA)

(73) Assignee: DANIÉLE HENKEL TO GO, Pierrefonds (CA)

(21) Appl. No.: 15/558,912

(22) PCT Filed: Oct. 27, 2015
(86) PCT No.: PCT/CA2015/051094

§ 371 Date: Sep. 15, 2017

Related U.S. Application Data
(60) Provisional application No. 62/133,703, filed on Mar. 16, 2015.

(51) Int. Cl. G06Q 30/02; A61Q 19/08; A61K 8/02

(52) U.S. Cl. CPC G06Q 30/0251; A45D 44/002; A61K 8/0212; A61Q 19/08

(57) ABSTRACT

The present disclosure relates to method and systems for carrying out various treatments, For example, the disclosure relates to a method for improving a client experience and/or occupying waiting times, while increasing and/or optimizing revenues for a business owner. The method comprises displaying a treatment space in a public space; advertising in the public space at least one type of body treatment; optionally creating or updating a client record for a given client; and applying the body treatment in the treatment space to at least one body area of the given client. For example, the disclosure also relates to system for providing body treatment, the system comprising a body treatment machine; a first seating apparatus having a reclined backing member; and a second seating apparatus positioned proximate a headrest portion of the first seating apparatus.

### CROSS REFERENCE TO RELATED APPLICATIONS

The present application claims priority to U.S. 62/133,703, filed on Mar. 16, 2015, that is hereby incorporated by reference in its entirety.

### FIELD OF THE DISCLOSURE

The present disclosure relates to a method and system for carrying out various treatments, and more particularly a method and system for carrying out body treatment in a public space.

### BACKGROUND OF THE DISCLOSURE

People now typically have busy schedules that require balancing work, family life, social life, as well as other regular tasks such as groceries, shopping and personal care. Multi-tasking becomes useful in completing a person's task.

Furthermore, aging is a concern for members of the public. Various products and treatments are available to combat the signs and effects of aging. One of the goals of these products and treatments is to reduce the signs of aging by improving the cosmetic appearance of a person.

### SUMMARY

It would thus be highly desirable to be provided with a device, system or method that would at least partially address the disadvantages of the existing technologies.

The embodiments described herein provide in one aspect a method for improving a client experience and/or occupying waiting times, while increasing and/or optimizing revenues for a business owner. The method comprises displaying a treatment space in a public space, advertising in the public space at least one type of body treatment, optionally creating or updating a client record for a given client; and applying the body treatment in the treatment space to at least one body area of the given client.

The embodiments described herein provide in another aspect a method for improving a client experience and/or occupying waiting times, while increasing and/or optimizing revenues for a business owner, the method comprising displaying a treatment space in a public space belonging to the business owner, the treatment space having located thereon a seating apparatus and a body treatment equipment, advertising in the public space at least one type of body treatment available to be carried out in the treatment space using the body treatment equipment, creating or updating a client record for a given client, receiving a selection of at least one body area of the given client to be treated, applying the body treatment in the treatment space using the body treatment equipment to the at least one body area of the given client, wherein the given client is visible from the public space as the client is receiving the body treatment, wherein creating or updating the client record comprises storing information related to the body treatment that is applied and wherein the public space comprises at least one service other than the body treatment that is at least offered to the client.

The embodiments described herein provide in another aspect a method for delivering a treatment, the method comprising receiving a selection of a body area to be treated, choosing a type of treatment based on the body area and at least one property of the body area, and applying a body treatment to the body area in a non-clinical setting.

The embodiments described herein provide in another aspect a system for providing a body treatment, the system comprising a body treatment machine, a first seating apparatus having a reclined backing member and a second seating apparatus positioned proximate a headrest portion of the first seating apparatus.

### DRAWINGS

The following drawings represent non-limitative examples in which:

FIG. 1A illustrates a flowchart of the steps of an exemplary method for providing a treatment;

FIG. 1B illustrates a flowchart of the steps of an exemplary method for providing a treatment;

FIG. 2 illustrates a schematic diagram of a computerized system for partially delivering a treatment according to one exemplary embodiment; and

FIG. 3 illustrates a perspective view of a treatment system according to one exemplary embodiment.

### DESCRIPTION OF VARIOUS EMBODIMENTS

The following examples are presented in a non-limiting manner.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

“Body treatment” as used herein refers to a treatment applied to a part of a human body with the assistance of a machine. For example, a body treatment may include skin treatment, muscle treatment, joint treatment and/or bone treatment.

“Skin treatment” as used herein refers to any treatment applied to the skin surface of human body with the assistance of machine. For example, the skin treatment may include anti-aging treatment, massaging treatment, tattoo removal, laser treatment, electroporation treatment, radiofrequency treatment, microdermabrasion treatment, microneedling treatment, peeling treatment, and skin burn, skin stimulation, and cellular stimulation.

“Anti-aging” as used herein refers to a property or character that is related to combatting the natural condition of aging. Anti-aging may refer to a cosmetic or superficial property, such a visible signs of aging. Anti-aging may also refer to a non-cosmetic property, such as improvements that are not immediately visible, such as cellular improvement or increase of collagen, elastin, and/or hyaluronic acid.

“Non-clinical setting” as used herein refers to a setting that is not primarily directed to the treatment and/or care of patients. It will understood that a non-clinical setting is in contrast to a “clinical setting”, which is a setting primarily directed to the treatment and/or care of patients, such as hospitals, medical clinic, healthcare centres, and specialist clinics, etc.

“Client” as used herein refers to a person who receives the body treatment.

According to example methods disclosed herein, the client record comprises subscription information and wherein storing the client profile comprises updating the subscription information based on the body treatment that is applied.

According to example methods disclosed herein, the method further comprising taking a photograph of the given client prior to applying the body treatment and taking a photograph of the given client after applying the body treatment.

According to example methods disclosed herein, creating or updating the client record comprises storing in the client record the photograph taken prior to applying the body treatment and the photograph taken after applying the body treatment.

According to example methods disclosed herein, further comprising applying a product to the body area after applying the treatment.

According to example methods disclosed herein, applying the product comprises applying a cream or lotion to the body area based on the body treatment that is applied.

According to example methods disclosed herein, the body area is chosen from a group consisting of facial area, neck area, collar area, feet area, hand area, leg area and shoulder area.

According to example methods disclosed herein, creating or updating the client record comprises creating an record entry indicating a further treatment, the record entry being created based on the body treatment that is applied.

According to example methods disclosed herein, the body treatment is applied to the given client while at least one other service provided to the given client is in a waiting phase.

According to example methods disclosed herein, the public space is a drug store and wherein the waiting phase of at least one other service provided to the client is filling an order for medication.

According to example methods disclosed herein, the public space is a transportation and wherein the waiting phase of at least one other service provided to the client is waiting for boarding a transportation service.

According to example methods disclosed herein, the public space is a clinic and wherein the waiting phase of the at least one other service provided to the client is a waiting period for receiving a treatment other than the body treatment.

According to example methods disclosed herein, the body treatment is an anti-aging treatment.

According to example methods disclosed herein, the anti-aging treatment is a skin stimulation treatment.

According to example methods disclosed herein, the treatment is applied for a duration of less than about 30 minutes.

According to example methods disclosed herein, the treatment is applied for a duration of less than about 10 minutes.

According to example methods disclosed herein, the treatment space is partially covered by an upstanding member positioned along an arc of a circumferential perimeter surrounding the treatment space.

According to example methods disclosed herein, the upstanding member is positioned at a position corresponding to the head of the client seated in the seating apparatus.

According to example methods disclosed herein, the method further includes providing a client record for a client whose body area is treated, recording in the client record the date of the treatment and the body area treated and, optionally recording a duration of the treatment.

According to example methods disclosed herein, the method further includes taking a photograph of the body area prior to the applying the treatment and taking a photograph of the body area after applying the treatment.

According to example methods disclosed herein, the non clinical setting is the retail space of a retail store.

According to example methods disclosed herein, the retail store is a drug store.

According to example methods disclosed herein, the public space is a waiting room of a clinic.

According to example methods disclosed herein, wherein the public space is a transportation centre (for example, bus station, port, airport, train station).

According to example methods disclosed herein, wherein the public space is a beauty care center.

According to example methods disclosed herein, wherein the public space is a beauty care center.

According to example methods disclosed herein, wherein the public space is a hairdresser center.

According to example methods disclosed herein, the at least one service other than the body treatment that is at least offered to the client can be any type of body treatment as described in the present disclosure.

According to example methods disclosed herein, the at least one service other than the body treatment that is at least offered to the client can be any type of service as discussed in the present disclosure or any type of service offered in the different locations recited in the present disclosure.

According to example methods disclosed herein, the at least one product that is at least offered to the client can be at least one product that is complementary to the at least one body treatment. For example, it can be a product to be used after the treatment (post-treatment). It can also be a product that is not related to the body treatment.

According to example methods disclosed herein, the body area being treated is at least partially visible from the public space.

According to example methods disclosed herein, a physical area of the treatment is partially covered by an upstanding member positioned along an arc of a circumferential perimeter surrounding the physical area.

According to example methods disclosed herein, the upstanding member is positioned at a position corresponding to the head of a client whose body area is treated.

According to example systems disclosed herein, the anti-aging treatment machine is a skin stimulation machine.

According to example systems disclosed herein, the system further includes an upstanding member extending upwardly and partially surrounding the first seating apparatus.

According to example systems disclosed herein, the upstanding member extends laterally to define a treatment space, at least the first seating apparatus and the second seating apparatus being positioned within the treatment space.

According to example systems disclosed herein, the upstanding member is positioned along an arc of a circumferential perimeter of the treatment space, the upstanding member partially obstructing the treatment space when viewed from one direction.

According to example systems disclosed herein, the upstanding member is located proximate the headrest portion of the first seating apparatus.

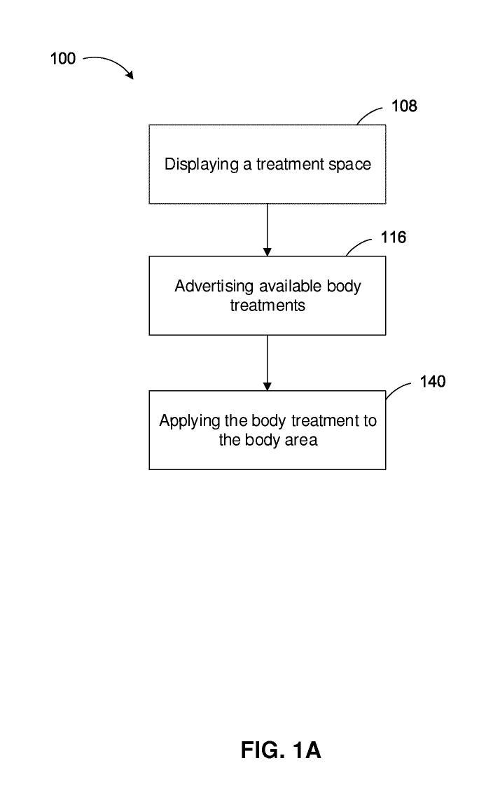

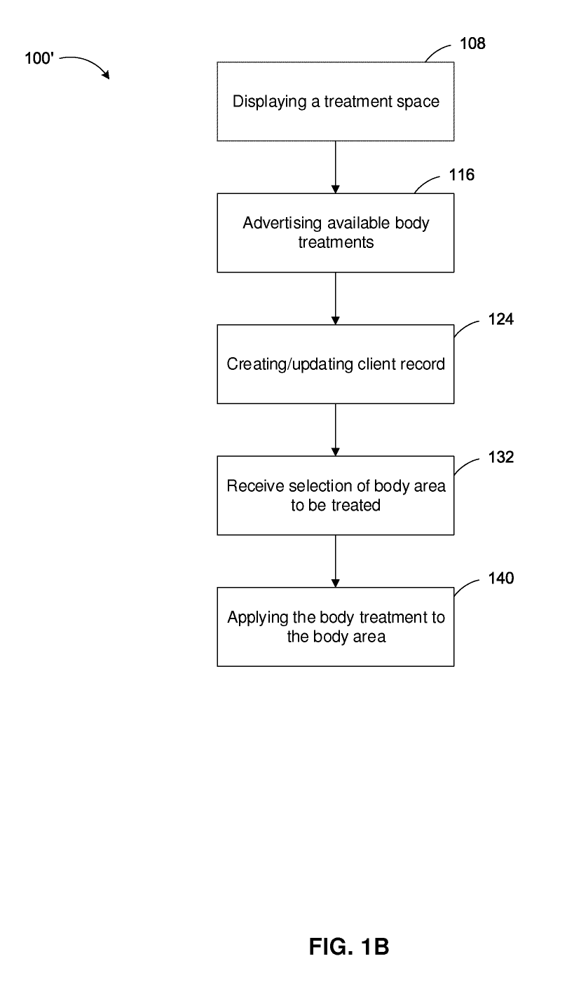

Referring now to FIG. 1A illustrated therein is a flowchart of the steps of an exemplary method 100 for providing a treatment. The treatment is provided in a way so as to improve client experience. The treatment may further be a way of occupying waiting times. Furthermore, a business offering the treatment can increase or optimize revenues of the business by providing the treatment.

At step 108, a treatment space is displayed in a public space belonging to a business owner. The treatment space has located thereon at least one seating apparatus and a body treatment equipment. The seating apparatus is adapted to seat a client who is to receive a body treatment. The body treatment is applied with the assistance of the body treatment equipment. By placing the treatment space in a public space, it becomes readily visible to patrons at the public space, thereby raising curiosity and awareness of the treatment space.

At step 116, a plurality of types of body treatment available to be carried out in the treatment space are advertised. Accordingly, a potential client or existing client may immediately appreciate the treatments that can be applied in the treatment space. For example, the advertising may be carried out using visible displays, such as posters or images/videos displayed on an electronic display. The advertising may also be carried out using pamphlets or other types of literature that may be handed out. The advertising may also be provided in an audio manner, such as through audio announcements made in the public space.

A client record may optionally be created or updated. A client record refers to a stored record that includes information pertaining to the client. Where the client record is for an existing client, updating the client record includes retrieving the stored record for that client. Where the client profile is a new client, a new record is created for the new client.

A client record includes identification information for the client, such as name and contact information. The client record further includes physical or physiological information for the client, such as age, sex, aging level, etc. Physical or physiological information may include photographs of one or more body areas of the client. The client record may further include treatment information for the client, such as date, type, and/or duration of previously received treatments. The client record may further include payment information for the client, such as prepaid credits and/or information entitling the client to further treatment.

Where a new client record is to be created, creating the record includes collecting one or more of the information included in a stored record. Identification information and physical or physiological information may be collected. Furthermore, a photograph of one or more body areas of the client may be taken. It will be appreciated that the photographs are taken prior to the new client receiving any anti-aging treatment.

At step 140, the body treatment is applied to a body area of the client according to the determined at least one treatment parameter. The body treatment is applied to the client in the public space. Consequently, the client receiving the body treatment is visible from the public space. For example, the client is visible to other patrons in the public space.

Referring now to FIG. 1 B, illustrated therein is a flowchart of the steps of an exemplary method 100′ for providing a treatment. The treatment is provided in a way so as to improve client experience. The treatment may further be a way of occupying waiting times. Furthermore, a business offering the treatment can increase or optimize revenues of the business by providing the treatment.

At step 108, a treatment space is displayed in a public space belonging to a business owner.

At step 116, a plurality of types of body treatment available to be carried out in the treatment space are advertised.

At step 124, a client record is created or updated.

At step 132, a selection of at least one body area to be treated is received. The selection may be based on physical or physiological information for the client.

For example, a selected body area may be an area having more apparent signs of aging.

For example, a selected body area may be a default selection, such as the facial area.

For example, a selected body area may be made based on previous treatments applied to the client (ex: if a client recently received treatment a first area, such as facial area, the selected body area may be a second area, such as hand area).

For example, a selected body area may also be a user-made choice, such a choice made by the client. For example, the client may choose to receive a treatment other than to the body area because the client has already applied make-up to the face and does not wish to remove the make-up.

Accordingly to various exemplary embodiments, the set of body areas available for selection at step 116 may be less than the full set of body areas that may be treated. For example, a body treatment equipment that is available may be adapted to treat a given set of body areas that the set of body areas made available for selection is smaller than that given set.

For example, the set of body areas made available for selection may correspond to body areas that are typically exposed (i.e. not covered by clothing, such as innerwear). Accordingly, a client does not need to remove additional pieces of clothing in order to allow access to these body areas. For example, the body areas made available for selection consists of facial area, subareas of the facial area, neck area, collar area, hand area, foot area, arm area, leg area, feet area and shoulder area. In other exemplary embodiments, the body areas made available for selection is a subset of the previously listed body areas.

At least one treatment parameter may be further determined. Treatment parameter herein refers to at least one parameter to be set when applying the body treatment to the client.

For example, in the context of anti-aging treatment, the at least one parameter may be a setting of the anti-aging equipment, whereby using different settings of the anti-aging equipment causes different treatment results. The at least one parameter may also be a parameter external to the anti-aging equipment, such as duration of the treatment. The at least one parameter may also be a product, such as lotion or cream, that is external to the anti-aging equipment to be applied as part of the anti-aging treatment.

At step 140, the body treatment is applied to the selected body area of the client according to the determined at least one treatment parameter.

For example, the body treatment (of method 100 or method 100′) may be an anti-aging treatment. The anti-aging treatment may be a skin stimulation treatment equipment. For example, the anti-aging treatment may be a skin stimulation treatment provided by the LPG™ company, such as the Mobilift M6™ system.

The body treatment may be applied with the assistance of a clerk (such as sales clerk) following predetermined guidelines for using the body treatment equipment. Accordingly a low level of training is required to operate the body treatment equipment. The skill required is far below a level required of professional skills.

The body treatment may be applied automatically using the body treatment equipment. For example, only configuration of the equipment, such as entering of predetermined parameters, is required prior to applying the body treatment. For example, the remainder of the body treatment carried out by the body treatment equipment is machine controlled (ex: computer controlled).

Advantageously, by offering body treatments that can be carried out by a clerk having a low level of training or by a machine control, the treatment may be offered at a lower cost to the client. This provides the perception that the body treatment is accessible to many clients. For example, by contrast, there is a preconception that many personal care treatments offered in specialized clinics are often very expensive and inaccessible.

Furthermore, by offering body treatments that can be carried out by a clerk having a low level of training or by a machine control, the treatment may be offered at a lower cost to the business owner, therefore contributing to better/higher profit margins for the business owner.

According to one exemplary embodiment, the body treatment may be applied for a duration of less than about 30 minutes. For example, the body treatment may be applied for a duration of less than about 10 minutes.

The exemplary method 100 or method 100′ may further include taking a photograph of the body area after applying the treatment. Taking a photograph of the body area prior to the treatment and taking a photograph after the treatment allows a client that received the anti-aging treatment to immediately compare the photographs and appreciate the effects of the treatment.

The public space may be a space that does not have the primary purpose of offering body treatments. For example, the public space may offer one or more other services that are unrelated or only incidentally related to the applying of body treatments.

The body treatment may further be applied while a client is simultaneously receiving another service within the public space. For example, the other service may be in a waiting phase. “Waiting phase” of an offered service herein refers to a phase of a service where the client must wait for the service provider to complete an action. Accordingly, the client must wait a waiting period. This waiting period may represent wasted time or sub-optimally used time for the client.

According to various exemplary embodiments, the treatment is applied in a retail setting. The retail setting may be a shopping mall, department store or drug store. It will be appreciated that by applying the treatment in a retail setting, the client will be visible to other patrons (i.e. third parties) of the retail space while receiving his or her anti-aging treatment.

It will be further appreciated that applying the treatment in a retail setting allows the treatment to be provided in an “on-the-go” manner. A client may receive the treatment as part of a bundle of other tasks carried out by the client within the retail setting. For example, in a retail setting, the client may receive the anti-aging treatment in addition to one or more other retailing actions (ex: purchasing one or more items, picking up an order for a prescription drug, dry cleaning, photographs, food, etc.). Advantageously, the client will have had an improved client experience in that he or she can occupy the waiting time of another service by receiving the body treatment during that waiting time. Advantageously, the business owner will have increased revenues in having a client consume another service while at the public space.

According to various exemplary embodiments, the public space may be a setting other than a retail setting. For example, the public space may be a transportation centre, such as a bus stop, train station or airport, and the waiting phase may be the waiting period for boarding a transportation service.

According to various exemplary embodiments, the public space may be a clinical setting and the waiting phase may be the waiting period (ex: the waiting room) for receiving a clinical treatment. The clinical setting may be one where specialized care is offered. The body treatment may be applied while waiting to receive the clinical treatment. For example, the clinical treatment may be a treatment offered by a dentist, doctor, ophthalmologist or similar medical specialist.

Furthermore, making the treatment visible to other patrons may act as a marketing tool for attracting new clients.

It will be appreciated that limiting the set of body areas that is treated allows the treatment to be applied to a client in a public space without embarrassment to the client. For example, by applying the body treatment to only the group of body areas that are typically exposed, the client can receive the treatment without feeling embarrassed.

According to various exemplary embodiments, the method 100 or 100′ further includes applying a cream or lotion to the body area. The type of cream or lotion that is applied may be chosen based on a condition of the body area that is treated. Additionally or alternatively, the type of cream or lotion that is applied may be chosen based the type of treatment or parameters of the treatment that was applied.

According to various exemplary embodiments, the method 100 or 100′ further includes updating the client record for the client receiving the treatment may creating a record entry that store information about the body treatment that is applied. For example, the type of treatment applied, the chosen parameters, and/or the duration may be entered.

Various portions of the method for carrying out the body treatment may be carried on a computer. The embodiments of the systems and methods described herein may be implemented, at least partially, in hardware or software, or a combination of both. For example, some embodiments may be implemented in computer systems and computer programs, which may be stored on a physical computer readable medium, executable on programmable computers (e.g. computing devices and/or processing devices) each comprising at least one processor, a data storage system (including volatile and non-volatile memory and/or storage elements), at least one input device (e.g. a keyboard or mouse), and at least one output device (e.g. a display screen, a network, or a remote server). For example and without limitation, the programmable computers may include servers, personal computers, laptops, netbook computers, personal data assistants (PDA), cell phones, smart phones, gaming devices, and other mobile devices.

In some embodiments, program code can be applied to input data to perform the functions described herein and to generate output information. The output information can then be supplied to one or more output devices for outputting to one or more users.

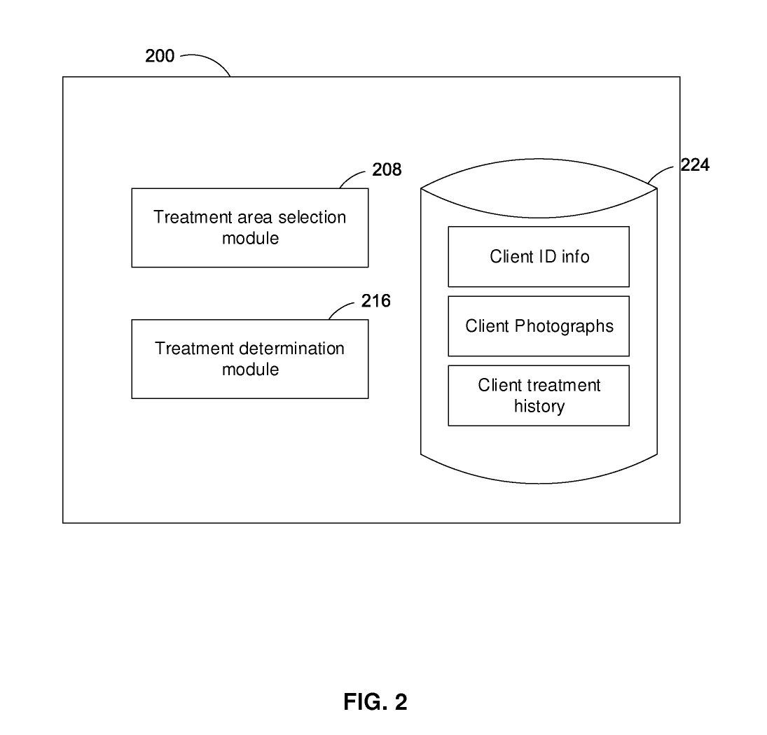

Referring now to FIG. 2, therein illustrated is a schematic diagram of a computerized system operable to carry out portions of the method 100 or method 100′ for providing a body treatment. The system includes a treatment area selection module 208 that is operable to receive the selection of the at least one body area to be treated. The system further includes a treatment determination module 216, which is operable to determine at least one treatment parameter for the body treatment to be carried out. The system further includes a storage device 224, which is operable to store a plurality information, such as client identification information, before and after photographs of clients, and the treatment history for clients.

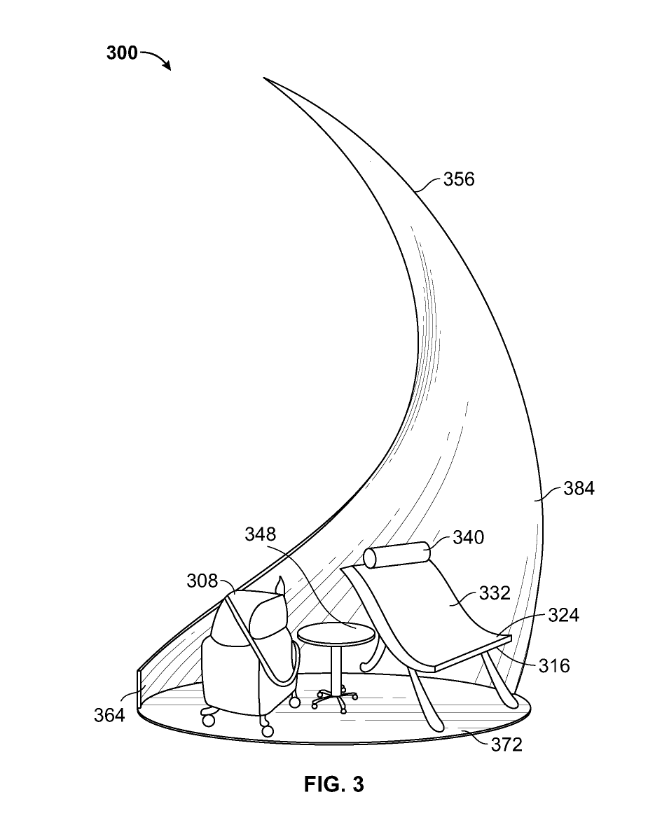

Referring now to FIG. 3, therein illustrated is a perspective view of a system 300 for providing an body treatment according to one exemplary embodiment. The system 300 includes body treatment equipment 308. The body treatment equipment 308 may be a mobile system, which allows it to be easily displaced. For example, the body treatment is an anti-aging treatment.

For example, the body treatment equipment 308 may be a skin stimulation treatment equipment. For example, the body treatment may be a skin stimulation treatment provided by the LPG™ company, such as the Mobilift M6™ system. The skin stimulation treatment may be applied with the assistance of a technician following predetermined guidelines for using the skin stimulation treatment equipment.

The system 300 further includes a seating apparatus 316. The seating apparatus 316 includes a seat portion 324 and a reclined backing portion 332 extending therefrom. The reclined backing portion 332 may further include a headrest portion 340. Accordingly, a user, such as a client, seated in the seating apparatus will assume a reclined position in which he or she will be facing substantially upwards. The reclined position provides easy access to the user's face for treatment.

The system 300 further includes a second seating apparatus 348. The second seating apparatus 348 is positioned proximate the headrest portion 340 of the first seating apparatus 316. Accordingly, a treatment technician may be seated on the second seating apparatus 348 while using the body treatment machine 308 to apply a body treatment to a client seated within the first seating apparatus 316.

Continuing with FIG. 3, the system 300 may further optionally includes an upstanding member 356 extending upwardly and partially surrounding the first seating apparatus 316. For example, and as illustrated, the upstanding member 356 includes a base portion 364 that extends laterally to define an arc. Continuing an arc defines a treatment space 372. The arc shape of the base portion 364 provides a sense of partially enveloping or encompassing the treatment space 232. Accordingly, the upstanding member 356 partially obstructs the treatment space 372 when viewed from a first direction, such as from behind the upstanding member 356. However, since the upstanding member 356 only partially surrounds the first seating apparatus 316 and the treatment space 372, the treatment space is viewable from a second direction that is opposite the first direction.

According to one exemplary embodiment, the treatment space 372 includes a substrate member coupled to the upstanding member 356. For example, the substrate member has a color that matches at least one color of the upstanding member 356, thereby giving the appearance of continuity between the upstanding member 356 and the substrate member defining the treatment space 372.

The upstanding member 356 further includes an intermediate portion 384. The intermediate portion 384 corresponds to a portion of the upstanding member 356 having a height that corresponds to the height portion of the headrest portion 340 of the first seating apparatus 316. For example, the intermediate portion 384 has a lateral width that is narrower than the base portion 364 of the upstanding member 356.

According to various exemplary embodiments, the upstanding member 356 and the first seating apparatus 316 are positioned relative to one another so that the upstanding member 356 is located proximate the headrest portion of the first seating apparatus. By placing the upstanding member 356, a client seated in the first seating apparatus 316 and resting his or head on the headrest portion 340 is provided an increased sense of intimacy. For example, the client feel as being in more intimate space in knowing that someone cannot see her from behind. Furthermore, by having the upstanding member 356 only partially surround the treatment space 372, the client also feels a sense of openness, such as around the lower body part. This imparts a feeling of accessibility to the treatment system 300.

According to various exemplary embodiments, the treatment system 300 may further include a display apparatus for holding and displaying a plurality of treatment products related to the treatment system 300, such as lotions or creams. These treatment products may be applied after applying the body treatment with the body treatment equipment 308.

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

1. A method for improving a client experience and/or occupying waiting times, while increasing and/or optimizing revenues for a business owner, the method comprising:
displaying a treatment space in a public space;
advertising in the public space at least one type of body treatment;
optionally creating or updating a client record for a given client; and
applying the body treatment in the treatment space to at least one body area of the given client.

2. A method for improving a client experience and/or occupying waiting times, while increasing and/or optimizing revenues for a business owner, the method comprising:
displaying a treatment space in a public space belonging to the business owner, the treatment space having located thereon a seating apparatus and a body treatment equipment;
advertising in the public space at least one type of body treatment available to be carried out in the treatment space using the body treatment equipment;
creating or updating a client record for a given client;
receiving a selection of at least one body area of the given client to be treated;
applying the body treatment in the treatment space using the body treatment equipment to the at least one body area of the given client;
wherein the given client is visible from the public space as the client is receiving the body treatment;
wherein creating or updating the client record comprises storing information related to the body treatment that is applied; and
wherein the public space comprises at least one service other than the body treatment that is at least offered to the client and/or at least one product that is at least offered to the client.

3. The method of claim 1 or 2, wherein the client record comprises subscription information and wherein storing the client profile comprises updating the subscription information based on the body treatment that is applied.

4. The method of any one of claims 2 to 3, wherein further comprising:
taking a photograph of the given client prior to applying the body treatment; and
taking a photograph of the given client after applying the body treatment.

5. The method of claim 4, wherein creating or updating the client record comprises storing in the client record the photograph taken prior to applying the body treatment and the photograph taken after applying the body treatment.

6. The method of any one of claims 2 to 5, further comprising applying a product to the body area after applying the treatment.

7. The method of claim 6, wherein applying the product comprises applying a cream or lotion to the body area based on the body treatment that is applied.

8. The method of any one of claims 2 to 7, wherein the body area is chosen from a group consisting of facial area, neck area, collar area, feet area, hand area, leg area and shoulder area.

9. The method of any one of claims 2 to 8, wherein creating or updating the client record comprises creating an record entry indicating a further treatment, the record entry being created based on the body treatment that is applied.

10. The method of any one of claims 2 to 9, wherein the the body treatment is applied to the given client while at least one other service provided to the given client is in a waiting phase.

11. The method of any one of claims 2 to 9, wherein the public space is a drug store and wherein the waiting phase of at least one other service provided to the client is filling an order for medication.

12. The method of any one of claims 2 to 9, wherein the public space is a transportation and wherein the waiting phase of at least one other service provided to the client is waiting for boarding a transportation service.

13. The method of any one of claims 2 to 9, wherein the public space is a clinic and wherein the waiting phase of the at least one other service provided to the client is a waiting period for receiving a treatment other than the body treatment.

14. The method of any one of claims 2 to 13, wherein the body treatment is an anti-aging treatment.

15. The method of claim 14, wherein the anti-aging treatment is a skin stimulation treatment.

16. The method of claim 14 or 15, wherein the treatment is applied for a duration of less than about 30 minutes.

17. The method of claim 14 or 15, wherein the treatment is applied for a duration of less than about 10 minutes.

18. The method of any one of claims 2 to 17, wherein the treatment space is partially covered by an upstanding member positioned along an arc of a circumferential perimeter surrounding the treatment space.

19. The method of claim 18, wherein the upstanding member is positioned at a position corresponding to the head of the client seated in the seating apparatus.

20. A method for delivering a treatment, the method comprising:
receiving a selection of a body area to be treated;
choosing a type of treatment based on the body area and at least one property of the body area; and
applying the treatment to the body area in a public space.

21. The method of claim 20, wherein the body area is chosen from a group consisting of facial area, neck area, collar area, feet area, hand area, leg area and shoulder area.

22. The method of claim 20 or 21, wherein the treatment is an anti-aging treatment.

23. The method of claim 22, wherein the anti-aging treatment is a skin stimulation treatment.

24. The method of any one of claims 20 to 23, wherein the treatment is applied for a duration of less than about 30 minutes.

25. The method of any one of claims 20 to 23, wherein the treatment is applied for a duration of less than about 10 minutes.

26. The method of any one of claims 20 to 25, further comprising:
providing a client record for a client whose body area is treated;
recording in the client record the date of the treatment and the body area treated; and
optionally recording a duration of the treatment.

27. The method of claim 26, further comprising:
taking a photograph of the body area prior to the applying the treatment; and
taking a photograph of the body area after applying the treatment.

28. The method of any one of claims 20 to 27, wherein the public space is the retail space of a retail store.

29. The method of claim 28, wherein the retail store is a drug store.

30. The method of any one of claims 20 to 27, wherein the public space is a transportation centre.

31. The method of any one of claims 20 to 27, wherein the public space is a waiting room of a clinic.

32. The method of claim 28 or 29, wherein the body area being treated is at least partially visible from the public space.

33. The method of claim 32, wherein a physical area of the treatment is partially covered by an upstanding member positioned along an arc of a circumferential perimeter surrounding the physical area.

34. The method of claim 33, wherein the upstanding member is positioned at a position corresponding to the head of a client whose body area is treated.

35. A system for providing body treatment, the system comprising:
a body treatment machine;
a first seating apparatus having a reclined backing member; and
a second seating apparatus positioned proximate a headrest portion of the first seating apparatus.

36. The system of claim 35, wherein the body treatment machine is an anti-aging machine.

37. The system of claim 36, wherein the anti-aging machine is a skin stimulation machine.

38. The system of any one of claims 35 to 37, further comprising:
an upstanding member extending upwardly and partially surrounding the first seating apparatus.

39. The system of claim 38, wherein the upstanding member extends laterally to define a treatment space, at least the first seating apparatus and the second seating apparatus being positioned within the treatment space.

40. The system of claim 39, wherein the upstanding member is positioned along an arc of a circumferential perimeter of the treatment space, the upstanding member partially obstructing the treatment space when viewed from one direction.

41. The system of claim 40, wherein the upstanding member is located proximate the headrest portion of the first seating apparatus.
